(19) United States
(12) Patent Application Publication
(10) Pub. No.: US 2005/0196444 A1
(43) Pub. Date: Sep. 8, 2005

(54) Aqueous polymer dispersions based on alkyl (meth)acrylates

(75) Inventors: Karl Kolter, Limburgerhof (DE); Maximilian Angel, Schifferstadt (DE)

Correspondence Address: CONNOLLY BOVE LODGE & HUTZ LLP, SUITE 800, 1990 M STREET NW, WASHINGTON, DC, 20036-3425, US

(73) Assignee: BASF AKTIENGESELLSCHAFT, Ludwigshafen (DE)

(21) Appl. No.: 11/068,772

(22) Filed: Mar. 2, 2005

(30) Foreign Application Priority Data
Mar. 5, 2004 (DE) 102 004 011 349.1

(51) Int. Cl. A61K 9/24; B01J 13/00

(52) U.S. Cl. 424/472; 427/2.14

(57) ABSTRACT

An aqueous polymer dispersion for use as binder and coating agent for pharmaceutical dosage forms, obtained by free-radical polymerization in an aqueous system in the presence of a polymerization initiator of
i) 60 to 99.95% by weight of at least one monomer selected from the group consisting of alkyl acrylates, alkyl methacrylates and vinyl esters of C₁-C₂₄ carboxylic acids,
ii) 0.05 to 5% by weight of a monoolefinically unsaturated C₃-C₈ carboxylic acid iii) 0 to 35% by weight of further free-radical polymerizable monomers, where the total of i), ii), and iii) equals 100% by weight, in the presence of 0.001 to 1.0% by weight, based on the total weight of the monomers, of an anionic emulsifier.

The invention relates to aqueous polymer dispersions with a reduced emulsifier content, to processes for their preparation and to their use as excipient for pharmaceutical dosage forms.

The fact that stabilizers in pharmaceutical dispersions have an adverse effect on film formation has long been known. Thus, it is stated in “Aqueous Polymeric coatings for Pharmaceutical Dosage Forms” 2nd Edition, Ed. James W. McGinity 1997, Marcel Dekker Inc., New York, pages 56-67, that surfactants and water-soluble additives have very disadvantageous effects on film formation and the properties of films. Sodium lauryl sulfate is mentioned in particular, which alters the structure, the mechanical and permeation properties of films because, during film formation, it migrates into small interstices between the polymer particles. In addition, sodium lauryl sulfate is thought to be responsible for the unwanted properties of pH-dependence of release and for alteration during storage. Hydrophilic compounds such as polyethoxylates may also have very disadvantageous effects on films. Thus, for example, phase separation, flocculation, increase in the rate of release of active ingredient, deterioration in the mechanical properties and similar unwanted phenomena occur.

WO 03/087180 discloses that such problems are to be avoided by using coating dispersions which no longer contain any excipients such as emulsifiers. Instead, compounds having emulsifier properties, such as, for example, ester of (meth)acrylic acid with polyethylene glycol are incorporated into the film-forming polymers.

DE 100 11 447 discloses that dispersions comprising nonylphenol ethoxylates as emulsifier have disadvantages. According to this prior art, the problem of stabilizing dispersions of methacrylate copolymers is to be solved by using nonionic emulsifiers with an HLB of from 15.7 to 16.2. However, the preferred dispersions still have a relatively high emulsifier content.

It is an object of the present invention to find dispersions which have a minimal emulsifier content but at the same time have good use properties.

We have found that this object is achieved by aqueous polymer dispersions obtained by free-radical polymerization in an aqueous system in the presence of a polymerization initiator
i) 60 to 99.95% by weight of at least one monomer selected from the group consisting of alkyl acrylates, alkyl methacrylates and vinyl esters of C₁-C₂₄ carboxylic acids,
ii) 0.05 to 5% by weight of a monoolefinically unsaturated C₃-C₈ carboxylic acid
iii) 0 to 35% by weight of further free-radical polymerizable monomers,
   where the total of i), ii), and iii) equals 100% by weight, in the presence of 0.001 to 1.0% by weight, based on the total weight of the monomers, of an anionic emulsifier.

Preferred polymers are obtained from
i) 95 to 99.95% by weight of at least one monomer selected from the group consisting of alkyl acrylates, alkyl methacrylates and vinyl esters of C₁-C₂₄ carboxylic acids,
ii) 0.05 to 5% by weight of a monoolefinically unsaturated C₃-C₈ carboxylic acid.

Free-radical polymerizable monomers i) suitable according to the invention are compounds from the group consisting of C₁-C₂₄-alkyl esters of acrylic acid and of methacrylic acid, and C₁-C₂₄-alkylvinyl esters.

Alkyl esters of acrylic acid and methacrylic acid which may be mentioned are branched or unbranched C₁-C₂₄-alkyl esters, preferably methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 2-ethylhexyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosyl, n-docosyl or n-tetracosyl ester.

Preferred representatives which may be mentioned for the abovementioned alkyl radicals are branched or unbranched C₁-C₁₂-, particularly preferably C₁-C₆-alkyl radicals. Ethyl acylate and methyl methacrylate are particularly suitable.

A preferred embodiment of the invention relates to polymers which comprise C₁-C₆-alkyl esters of acrylic acid and/or methacrylic acid as monomers i).

A further preferred embodiment relates to polymers which comprise in each case an alkyl acrylate and an alkyl methacrylate.

Further suitable monomers i) are vinyl esters. Suitable vinyl esters of aliphatic C₁-C₂₄ carboxylic acids are vinyl esters of aliphatic branched or unbranched, saturated or unsaturated C₁-C₂₄ carboxylic acids. Examples which may be mentioned are formic acid, acetic acid, propionic acid, butyric acid, valeric acid, isovaleric acid, caproic acid, caprylic acid, capric acid, undecylenic acid, lauric acid, myristic acid, palmitic acid, palmitoleic acid, stearic acid, oleic acid, arachic acid, behenic acid and lignoceric acid.

Vinyl esters of the abovementioned C₁-C₁₂ carboxylic acids, in particular of the C₁-C₆ carboxylic acids, are preferably used. Vinyl acetate is very particularly preferred.

Suitable monomers ii) are monoethylenically unsaturated carboxylic acids having 3 to 8 C atoms, for example acrylic acid, methacrylic acid, dimethacrylic acid, ethacrylic acid, allylacetic acid, vinylacetic acid, crotonic acid and maleic acid. Acrylic acid and methacrylic acid are preferred monomers ii).

Suitable as further monomers iii) are amides of monoethylenically unsaturated carboxylic acids having 3 to 8 C atoms, such as, for example, amides of acrylic acid, methacrylic acid, dimethacrylic acid, ethacrylic acid, allylacetic acid, vinylacetic acid or crotonic acid, with preference for acrylic acid and/or methacrylic acid from this group of carboxylic acids.

Preferred representatives of this group which may be mentioned are acrylamide (H₂C═CH—CO—NH₂) and methacrylamide (CH₂═C(CH₃)—CO—NH₂). Suitable as further monomers iii) are also C₁-C₂₄-alkyl vinyl ethers, preferably C₁-C₁₂-alkyl vinyl ethers, N—C₁-C₂₄-alkyl-substituted amides of monoethylenically unsaturated C₃-C₈ carboxylic acids, N,N-C₁-C₂₄-dialkyl-substituted amides of monoethylenically unsaturated C₃-C₈ carboxylic acids, N-vinyllactams, N-vinylamines, styrene and butadiene.

Preferred C₁-C₁₂-alkyl radicals which may be mentioned for the vinyl ethers are branched or unbranched alkyl chains such as, for example, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 2-ethylhexyl, n-octyl, 2-ethylhexyl, n-nonyl, n-decyl, n-undecyl and n-dodecyl radicals.

It is possible to employ as further monomers iii) N—C₁-C₂₄-alkyl- or N,N-C₁-C₂₄-dialkyl-substituted amides of monoethylenically unsaturated C₃-C₈-carboxylic acids, where the alkyl radicals are branched or unbranched aliphatic alkyl radicals having 1 to 24 carbon atoms, for example methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 2-ethylhexyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosyl, n-docosyl or n-tetracosyl radicals, preferably alkyl radicals having 1 to 12, particularly preferably having 1 to 6, carbon atoms.

Examples of preferred amidated comonomers are N-methylacrylamide, N-ethyl-acrylamide, N-n-propylacrylamide, N-1-methylethylacrylamide, N-n-butylacrylamide, N-1-methylpropylacrylamide, N-2-methylpropylacrylamide, N-1,1-dimethyl-ethylacrylamide, N-n-pentylacrylamide, N-n-hexylacrylamide, N-methylmeth-acrylamide, N-ethylmethacrylamide, N-n-propylmethacrylamide, N-1-methylethylmethacrylamide, N-n-butylmethacrylamide, N-1-methyl-propylmethacrylamid, N-2-methylpropylmethacrylamid, N-1,1-dimethylethyl-methacrylamide, N-n-pentylmethacrylamide, N-n-hexylmethacrylamide.

Compounds which may be mentioned as N-vinyllactams or N-vinylamines are selected from the group consisting of N-vinylpyrrolidone, N-vinylpiperidone, N-vinylcaprolactam, N-vinylimidazole, methylated N-vinylimidazole and N-vinylformamide.

It is, of course, also possible to polymerize mixtures of each of the abovementioned monomers.

The content of monomers iii) is preferably zero.

Very particularly preferred polymers are obtained from
i) 95 to 99.95% by weight of ethyl acrylate or methyl methacrylate or mixtures thereof, and
ii) 0.05 to 5% by weight of methacrylic acid.

The ratio of alkyl acrylates to alkyl methacrylates is from 9:1 to 1:3, preferably 3:1 to 1:1, particularly preferably 2.5:1 to 1.5:1, by weight.

The emulsifiers used are anionic compounds, preferably anionic emulsifiers whose HLB is in the range >20. The HLB is a measure introduced by Griffin for the hydrophilicity or lipophilicity and means hydrophilic-lipophilic balance. This value can be found only by calculation for anionic emulsifiers.

A list of the appropriate compounds is to be found for example in Fiedler's Handbuch der pharmazeutischen und kosmetischen Hilfsstoffe, 4th edition, 1996, pages 82, 84.

Suitable anionic emulsifiers are:
ammonium salts or alkali metal, especially sodium and potassium salts, of C₈-C₃₀ fatty acids, alkyl sulfates, alkylsulfonates or alkylarylsulfonates, where the alkyl radicals have 8 to 30, preferably 10 to 24, C atoms, also corresponding salts of sulfosuccinic monoesters and diesters or of sulfated fatty alcohol ethoxylates.

Preferred anionic emulsifiers are sodium lauryl sulfate, sodium dioctyl sulfosuccinate, sodium stearate and sodium laurylsulfonate.

The emulsifiers are employed in amounts of from 0.001 to 1.0% by weight, in particular 0.005 to 0.95% by weight, preferably 0.01 to 0.5% by weight, particularly preferably 0.05 to 0.35% by weight, based on the total amount of monomers employed.

The polymers can be prepared by polymerizing the monomers in a manner known per se both with the aid of free radical-forming initiators and through the action of high-energy radiation, which is also intended to mean the action of high-energy electrons.

Suitable polymerization initiators are inter alia organic peroxides and hydroperoxides such as diacetyl peroxide, dibenzoyl peroxide, succinyl peroxide, di-tert-butyl peroxide, tert-butyl perbenzoate, tert-butyl perpivalate, tert-butyl permaleate, cumene hydroperoxide, diisopropyl peroxydicarbonate, bis(o-toluoyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide; inorganic peroxy compounds such as alkali metal peroxodisulfates, ammonium peroxodisulfate or H₂O₂; redox initiators such as H₂O₂/ascorbic acid, H₂O₂/Fe²⁺, peroxodisulfates/thiosulfates, peroxides/thiosulfates; azo initiators such as 4,4′-azobisisobutyronitrile, and mixtures of said initiators.

Preferred representatives of the abovementioned polymerization initiators are ammonium peroxodisulfate, the acidic alkali metal peroxodisulfates, especially the sodium and potassium salts, and the redox initiators H₂0₂/ascorbic acid.

The amounts of initiator or initiator mixtures used, based on monomer employed, are between 0.01 and 10% by weight, preferably between 0.1 and 1.5% by weight, particularly preferably between 0.1 and 0.8% by weight.

It may be advisable on use of preponderant amounts of vinyl esters as monomers i) to add a protective colloid.

The polymerization of the invention can be carried out continuously or discontinuously. A semi-discontinuous process is preferably used, in which for example a mixture of one part of polymerization initiator, emulsifier, buffer substance and monomer is heated to the polymerization temperature and, after the polymerization starts, the remainder, i.e. in each case polymerization initiator and monomer, is metered in simultaneously through separate feeds.

The polymerization takes place in the temperature range from 40 to 200° C., preferably in the range from 50 to 140° C., particularly preferably in the range from 60 to 100C. It is normally carried out under atmospheric pressure but can also take place under reduced or elevated pressure, preferably between 1 and 5 bar.

The reaction times are normally from 1 to 10, usually 1.5 to 5, hours, depending on the batch size.

To prepare storage-stable, aqueous polymer dispersions it is additionally advantageous, after the reaction of the polymerizable monomers is complete, to adjust the system to a pH in the range from 4 to 8, preferably in the range from 5 to 7. The pH adjustment can take place in manner known per se, for example by adding aqueous alkali metal hydroxide solutions.

The solids content of the resulting aqueous polymer dispersions or solutions is ordinarily from 10 to 70% by weight, preferably 15 to 65% by weight, particularly preferably 20 to 60% by weight.

The average molecular weight of the polymers (weight average), determined by light scattering, is from 300 000 to 1 000 000.

The aqueous polymer dispersions can be converted into the powder form by various drying processes such as, for example, spray drying, fluidized spray drying, drum drying or freeze drying. Owing to the advantageous low viscosity of the polymer dispersions, spray drying is preferably employed as drying process.

Where necessary, it is also possible in this case to add spraying aids such as, for example, colloidal silica, silicates, starch or starch derivatives, cellulose or cellulose derivatives, polyvinylpyrrolidones or polyvinyl alcohols.

An aqueous dispersion or solution can be prepared and used by redispersion in water of the dry polymer powder obtained in this way. The conversion into powder form has the advantage of easier possibility of transport, and a smaller tendency to microbial attack.

The invention therefore also relates to polymer powders prepared by drying aqueous polymer dispersions produced by the process of the invention.

The polymers of the invention are outstandingly suitable as release-slowing polymers for delaying release of active ingredient.

A preferred area of use of the water-dispersible polymers is the use as coating agent for solid pharmaceutical dosage forms.

Owing to the enormous flexibility and the low viscosity, when used as coating agents ordinarily no additional plasticizers are necessary.

The invention additionally relates to pharmaceutical dosage forms comprising at least one water-dispersible or water-insoluble polymer as coating agent and/or film-forming excipient, where the polymer, which can be employed both as aqueous dispersion and as polymer powder, is obtainable by the process described at the outset.

The coated dosage forms are preferably inter alia film-coated tablets, film-coated microtablets, sugar-coated tablets, coated pastilles, capsules, crystals, granules or pellets.

Active pharmaceutical ingredients which can be employed according to the invention are, besides medicinal substances, also vitamins, food supplements or dietetic active ingredients.

The dosage forms show delayed release of the active ingredient. Such forms are also referred to as sustained release forms. Sustained means in this connection that release takes place over a period of from 4 to 36 hours and that release of 80% of the active ingredient has taken place after 4 hours at the earliest. Sustained forms thus differ from fast-release forms with which 80% of the active ingredient is released after one hour at the latest.

Release from the forms is pH-independent. Release which is pH independent meant that release in simulated gastric fluid ( (0.1 N HCl; pH 1.2) does not differ from release in simulated intestinal fluid (phosphate buffer; pH 6.8). This means that the difference in release should not be more than 10% at any time during the test.

For use for pharmaceutical dosage forms, in addition it is possible to add to the dispersions conventional pharmaceutically acceptable excipients selected from the group consisting of antifoams, fillers, colors, pigments, antioxidants, preservatives, gloss improvers and plasticizers in the amounts customary for this purpose. It may be advisable with active ingredients of low solubility additionally to add a pore former to control the release. Examples of suitable pore formers are hydroxypropyl-methylcellulose, polyvinyl alcohol/polyethylene glycol graft copolymers such as Kollicoat® IR, polyvinyl alcohols, vinylpyrrolidone homopolymers and copolymers, natural polysaccharides, or low molecular weight water-soluble substances such as sugars, sugar alcohols, inorganic salts. Such pore formers may be present, depending on the solubility, in amounts of from 1 to 30, preferably 2 to 20,% by weight based on film former.

The total content of excipients can be up to 50% by weight based on the solids content of the dispersion.

It is also possible additionally to employ water-insoluble but water-swellable substances in small particle size such as microcrystalline cellulose, crosslinked sodium carboxymethylcellulose or carboxymethylstarch and crosslinked polyvinylpyrrolidone.

A particular advantage of the aqueous dispersions is, inter alia, their low tendency to foaming and thus reduced tendency to pitting. The dispersants additionally display very good stability, as is evident from the shear stability and sedimentation stability. The coatings obtained according to the invention display further advantages besides the low emulsifier content. Thus, the tackiness of the films is reduced in relation to that of comparable commercial compositions. In addition, the coatings display excellent mechanical properties.

The preparation of the aqueous polymer dispersions of the invention, and the use thereof, is explained in more detail in the following examples.

Preparation of the Polymers

General Method

The polymerization vessel was flushed with nitrogen. The deionized water was put into the polymerization vessel while stirring (120 rpm). The sodium lauryl sulfate (as 15% aqueous solution) was then added, washing with deionized water. The initial charge was heated to an internal temperature of 80° C. When an internal temperature of 70° C. was reached, feed 1 was rapidly added. At 75° C., feed 2 (7% by weight aqueous sodium peroxodisulfate solution) was rapidly added. At 80° C., feeds 3 (emulsion feed) and 4 (2.5% by weight aqueous sodium peroxodisulfate solution) were started. Feeds 3 and 4 were added over a period of 2 hours. After feeds 3 and 4 were complete, polymerization was continued for two hours. After the polymerization was complete, the polymerization mixture was cooled to 20° C. Feed 5 was then added, mixing for minutes.

### EXAMPLE 1

A dispersion with an EA/MMA/MM monomer ratio of 65.6/33.9/0.5% by weight was prepared by the general method. The emulsifier content was 0.3% by weight based on the weight of monomers.

| Initial | | |
|---|---|---|
| charge | | |
| 413.0 | g | deionized water |
| 3.85 | g | sodium lauryl sulfate (15% by weight aqueous solution) |
| 5.10 | g | deionized water |
| Feed 01 | | |
| 45.00 | g | of feed 3 |
| Feed 02 | | |
| 6.35 | g | sodium peroxodisulfate (7% by weight aqueous solution) |
| Feed 03 | | |
| 413.6 | g | deionized water |
| 5.0 | g | sodium lauryl sulfate (15% by weight aqueous solution) |
| 290.8 | g | ethyl acrylate |
| 150.3 | g | methyl methacrylate |
| 2.2 | g | methacrylic acid |
| 7.6 | g | deionized water |
| Feed 04 | | |
| 17.8 | g | sodium peroxodisulfate (2.5% by weight aqueous solution) |
| Feed 05 | | |
| 125.2 | g | deionized water |
| 3.55 | g | sodium hydroxide solution (10% by weight aqueous solution) |

The bluish white aqueous dispersion had a solids content of 31% by weight. The average particle size was 149 nm.

### EXAMPLE 2

Dispersion with an EA/MMA/MM monomer ratio of 66/33.9/ 0.1% by weight. The emulsifier content was 0.3% by weight based on monomers.

The initial weight of monomers was

| | | |
|---|---|---|
| 292.0 | g | ethyl acrylate |
| 150.9 | g | methyl methacrylate |
| 0.44 | g | methacrylic acid |

The bluish white aqueous dispersion had a solids content of 31% by weight. The average particle size was 141 nm.

### EXAMPLE 3

Exemplary dispersion with an EA/MMA/MM monomer ratio of 65.813410.2% by weight. The emulsifier content was 0.3% by weight based on monomers.

The initial weight of monomers was

| | | |
|---|---|---|
| 291.7 | g | ethyl acrylate |
| 150.7 | g | methyl methacrylate |
| 0.88 | g | methacrylic acid |

The bluish white aqueous dispersion had a solids content of 31% by weight. The average particle size was 154 nm.

### EXAMPLE 4

A dispersion with an EA/MMA/MM monomer ratio of 64/3214% by weight and with an emulsifier content of 0.3% by weight based on the weight of monomers can be prepared analogously.

Initial weight of monomers:

| | | |
|---|---|---|
| 284.0 | g | ethyl acrylate |
| 142.0 | g | methyl methacrylate |
| 18.0 | g | methacrylic acid |

The pH is adjusted to pH 7 with 3% by weight aqueous sodium hydroxide solution.

### EXAMPLE 5

A polymer with an EA/MMA/MM monomer ratio of 66/33.7/0.3% by weight and with an emulsifier content of 0.3% by weight based on the weight of monomers is prepared in analogy to example 1.

Initial weight of monomers:

| | | |
|---|---|---|
| 292.8 | g | ethyl acrylate |
| 144.5 | g | methyl methacrylate |
| 1.32 | g | methacrylic acid |

The pH is adjusted to pH 7 with 3% by weight aqueous sodium hydroxide solution.

Use examples

Unless indicated otherwise, all % data refer to % by weight.

### EXAMPLE 6

1.0 kg of propranolol HCl pellets (20% by weight propranolol HCl, 51.7% microcrystalline cellulose, 25.8% lactose, 2.5% vinylpyrrolidone/vinyl acetate (6:4) copolymer) with a particle size of 0.8-1.5 mm prepared by wet extrusion and subsequent spheronization were coated in a Glatt GPC G3 fluidized bed coater with the following coating formula:

| | | |
|---|---|---|
| Ethyl acrylate/methyl methacrylate copolymer dispersion | 300.0 | g |
| with 0.1% methacrylic acid from example 2 | | |
| Talc | 40.0 | g |
| Simethicone | 0.5 | g |
| Water | 190.0 | g |

The coating formula was prepared by stirring simethicone and talc into 190.0 g of water and dispersing by means of an Ultra-turrax at 8000 rpm for 10 min. This preparation was slowly stirred into the dispersion. After stirring for a further 30 min, the coating suspension was ready for use.

The coating suspension was applied at an inlet air temperature of 38° C and a spraying rate of 13 g/min by means of a Wurster insert onto the pellets in a fluidized bed. Completion of the spraying process was followed by drying at an inlet air temperature of 38° C. for a further 5 minutes. Release of the active ingredient was determined in a USP 23 paddle apparatus (Pharmatest PTW), there being release both in 0.1N HCl and in phosphate buffer of pH 6.8.

The results for the release were as follows:

| Release after | | 0.1N HCl (pH 1.2) | Phosphate buffer of pH 6.8 |
|---|---|---|---|
| 1 | h | 1.4% | 1.6% |
| 2 | h | 5.6% | 5.9% |
| 4 | h | 27.4% | 28.5% |
| 8 | h | 65.3% | 65.1% |
| 12 | h | 84.8% | 84.1% |
| 16 | h | 99.8% | 100.3% |

The release was completely pH-independent.

### EXAMPLE 7

1.0 kg of caffeine pellets (20% caffeine, 55.0% microcrystalline cellulose, 22.5% lactose, 2.5% vinylpyrrolidone/vinyl acetate (6:4) copolymer) with a particle size of 0.7-1.6 mm prepared by wet extrusion and subsequent spheronization were coated in a Glatt GPC G3 fluidized bed coater with the following coating formula:

| | | |
|---|---|---|
| Copolymer dispersion with 0.2% methacrylic acid from | 400.0 | g |
| example 3 | | |
| Hydroxypropylmethylcellulose 3 mPas | 12.0 | g |
| Talc | 40.0 | g |
| Simethicone | 0.5 | g |
| Water | 340.0 | g |

The coating formula was prepared by stirring hydroxypropylmethylcellulose, simethicone and talc into 340.0 g of water and dispersing by means of an Ultra-turrax at 8000 rpm for 15 min. This preparation was slowly stirred into the ethyl acrylate/methyl methacrylate copolymer dispersion from example 3. After stirring for a further 30 min, the coating suspension was ready for use.

The coating suspension was applied at an inlet air temperature of 42° C. and a spraying rate of 16 g/min by means of a Wurster insert onto the pellets in a fluidized bed. Completion of the spraying process was followed by drying at an inlet air temperature of 42° C. for a further 5 minutes. Release of the active ingredient was determined in a USP 23 paddle apparatus (Pharmatest PTW), there being release both in 0.1 N HCl and in phosphate buffer of pH 6.8.

The results for the release were as follows:

| Release after | | 0.1N HCl (pH 1.2) | Phosphate buffer of pH 6.8 |
|---|---|---|---|
| 1 | h | 1.0% | 0.8% |
| 2 | h | 4.5% | 4.3% |
| 4 | h | 11.7% | 11.9% |
| 8 | h | 31.3% | 31.1% |
| 12 | h | 53.6% | 52.9% |
| 16 | h | 72.8% | 72.1% |
| 20 | h | 87.2% | 86.3% |
| 24 | h | 99.4% | 98.1% |

The release was completely pH-independent.

### EXAMPLE 8

2.0 kg of theophylline pellets (60% theophylline, 37.5% microcrystalline cellulose, 2.5% vinylpyrrolidone/vinyl acetate (6:4) copolymer) with a particle size of 0.7-1.4 mm prepared by wet extrusion and subsequent spheronization were coated in a Glatt GPC G3 fluidized bed coater with the following coating formula:

| | | |
|---|---|---|
| Dispersion of example 3 | 400.0 | g |
| Microcristalline cellulose | 10.0 | g |
| Titanium dioxide | 10.0 | g |
| Kaolin | 10.0 | g |
| Simethicone | 1.0 | g |
| Water | 300.0 | g |

The coating formula was prepared by stirring simethicone, microcrystalline cellulose, titanium dioxide and kaolin into 300.0 g of water and dispersing by means of an Ultra-turrax at 8000 rpm for 10 min. This preparation was slowly stirred into the 30% copolymer dispersion. After stirring for a further 30 min, the coating suspension was ready for use.

The coating suspension was applied at an inlet air temperature of 41° C. and a spraying rate of 18 g/min by means of a Wurster insert onto the pellets in a fluidized bed. Completion of the spraying process was followed by drying at an inlet air temperature of 41° C. for a further 5 minutes. Release of the active ingredient was determined in a USP 23 paddle apparatus (Pharmatest PTW), there being release both in 0.1N HCl and in phosphate buffer of pH 6.8 for 2 h.

A test was carried out in parallel with the same formula but using Kollicoat EMM 30 D (2:1 ethyl acrylate/methyl methacrylate copolymer comprising 1.5% by weight ethoxylated nonylphenol based on the 30% strength aqueous dispersion), likewise determining the release.

The results for the release were as follows:

| Release after | | Dispersion of example 3 | Kollicoat EMM 30 D |
|---|---|---|---|
| 1 | h | 0.8% | 1.6% |
| 2 | h | 3.6% | 5.9% |
| 4 | h | 11.4% | 17.5% |
| 8 | h | 25.9% | 38.2% |
| 12 | h | 48.6% | 61.2% |
| 16 | h | 64.2% | 81.3% |
| 20 | h | 81.5% | 96.6% |
| 24 | h | 96.5% | 101.1% |

Standard deviation of the values for the release from 12 individual releases:

| | |
|---|---|
| Dispersion of example 3: | 3.1% |
| Kollicoat EMM 30 D: | 5.9% |

The release from the product of the invention was distinctly slower than that with Kollicoat EMM 30 D (with ethoxylated nonylphenol).

In addition, the reproducibility of release was distinctly better with the dispersion of the invention.

### EXAMPLE 9

Firstly, ambroxol HCl pellets (10% ambroxol HCl, 80.0% sucrose, 10.0% vinylpyrrolidone/vinyl acetate (6:4) copolymer) were prepared by drug layering of sucrose pellets with a particle size of 0.7-0.9 mm. This was done by spraying a solution of 7.5% vinylpyrrolidone/vinyl acetate (6:4) copolymer and 7.5% ambroxol HCl in water onto the sugar pellets in a fluidized bed coater until the pellets had an active ingredient content of 10%. 1.0 kg of these active ingredient pellets were subsequently coated in an Aeromatic Strea 1 fluidized bed coater with the following coating formula:

| | | |
|---|---|---|
| Dispersion of example 1 | 370.0 | g |
| Iron oxide | 8.0 | g |
| Titanium dioxide | 10.0 | g |
| Polyvinyl alcohol/polyethylene glycol | 6.0 | |
| graft copolymer (Kollicoat IR) | | |
| Simethicone | 0.5 | g |
| Water | 300.0 | g |

The coating formula was prepared by stirring iron oxide, simethicone, polyvinyl/alcohol/polyethylene glycol graft copolymer and titanium dioxide into 300.0 g of water and dispersing by means of an Ultra-turrax at 8000 rpm for 8 min. This preparation was slowly stirred into the dispersion of example 1. After stirring for a further 30 min, the coating suspension was ready for use.

The coating suspension was applied at an inlet air temperature of 40° C. and a spraying rate of 12 g/min by the top spraying method onto the pellets in a fluidized bed. Completion of the spraying process was followed by drying at an inlet air temperature of 40° C. for a further 5 minutes.

Release of the active ingredient was determined in a USP 23 paddle apparatus (Pharmatest PTW) with release in phosphate buffer pH 7.4.

The results for the release were as follows:

| | |
|---|---|
| Release after | Amount released |

| | | |
|---|---|---|
| 1 | h | 3.6% |
| 2 | h | 8.3% |
| 4 | h | 20.5% |
| 8 | h | 42.6% |
| 12 | h | 61.6% |
| 16 | h | 78.2% |
| 20 | h | 95.0% |

### EXAMPLE 10

Sedimentation Stability

Determined in a centrifuge at 4000 rpm equivalent to 2750 g (2750 times the acceleration of gravity) with the container being filled to the 80 mm level. The residue remaining after careful emptying of the container was read off in mm.

Sediment height [mm]

| Dispersion of | after 90 min | after 150 min |
|---|---|---|
| Kollicoat EMM 30 D | 7 | 9 |
| Example 2 | 6 | 7 |
| (0.1% methacrylic acid) | | |
| Example 3 | 5 | 7 |
| (0.2% methacrylic acid) | | |
| Example 1 | 5 | 6 |
| (0.5% methacrylic acid) | | |

The dispersions of the invention display a better sedimentation stability than the comparative product Kollicoat EMM 30 D with ethoxylated nonylphenol. The dispersions of the invention are more stable on storage.

### EXAMPLE 11

Shear Stability

The shear stability was determined using a cam stirrer having 8 stirring pins and 4 flow holes. This cam stirrer is immersed in the dispersion and rotates at 2000 rpm for 15 min. The dispersion is then passed through a 125 μm screen and washed, and the residue is determined after drying at 105° C.

| Dispersion | Coagulation [%] |
|---|---|
| Kollicoat EMM 30 D | 4.6 |
| Example 2 (0.1% methacrylic acid) | 0 |
| Example 3 (0.2% methacrylic acid) | 0 |
| Example 1 (0.5% methacrylic acid) | 0 |

The dispersions of the invention are considerably more stable to shear stresses than is the comparative product Kollicoat EMM 30D with ethoxylated nonylphenol. No coagulation therefore occurs during use for coating dosage forms.

### EXAMPLE 12

Tackiness of ethyl acrylate/methyl methacrylate (2:1) copolymer films determined by the Hoessel method

The Hoessel method is described in Cosmetics and Toiletries, 111 (8),73-77 (1996). This entails a carbon plunger being pressed on the film, and the blackening of the film is a measure of the tackiness of the film.

Assessment scale:

| | |
|---|---|
| very tacky | 5 |
| not tacky | 0 |

| | Tackiness at 20° C./80% |
|---|---|
| Dispersion | rel. humidity. |
| Kollicoat EMM 30 D | 3.25 |
| Example 2 (0.1% methacrylic acid) | 2.75 |
| Example 3 (0.2% methacrylic acid) | 2.5 |
| Example 1 (0.5% methacrylic acid) | 2.25 |

The tackiness of the films of the invention is distinctly less than that of the comparative product Kollicoat EMM 30 D with ethoxylated nonylphenol.

### EXAMPLE 13

Mechanical properties of ethyl acrylate/methyl methacrylate (2:1) copolymer films

The modulus of elasticity and elongation at break were determined using a TA-XT2 HiR texture analyzer from Stable Microsystems after the films had been conditioned at 23° C./54% relative humidity.

| | Modulus of | Elongation |
|---|---|---|
| Dispersion | elasticity [MPa] | at break [%] |
| Kollicoat EMM 30 D | 16 | >450 |
| Example 2 | 45 | >450 |
| (0.1% methacrylic acid) | | |
| Example 3 | 48 | >450 |
| (0.2% methacrylic acid) | | |
| Example 1 | 51 | >450 |
| (0.5% methacrylic acid) | | |

The films of the invention are as flexible as those of the comparative product Kollicoat EMM 30 D with ethoxylated nonylphenol, but are distinctly harder, as is evident from the higher modulus of elasticity. Harder coatings are advantageous in use because they are not so quickly damaged by mechanical stress.

1. An aqueous polymer dispersion for use as binder and coating agent for pharmaceutical dosage forms, obtained by free-radical polymerization in an aqueous system in the presence of a polymerization initiator, the aqueous polymer dispersion comprising a monomer mixture consisting essentially of:
i) 60 to 99.95% by weight of at least one monomer selected from the group consisting of alkyl acrylates, alkyl methacrylates and vinyl esters of C₁-C₂₄ carboxylic acids,
ii) 0.05 to 5% by weight of a monoolefinically unsaturated C₃-C₈ carboxylic acids and
iii) 0 to 35% by weight of free-radical polymerizable monomers, and 0.001 to 1.0% by weight of an anionic emulsifier, based on the total weight of the monomer mixture.

2. A dispersion according to claim 1, where the monomer mixture consists essentially of:
i) 95 to 99.95% by weight of at least one monomer selected from the group consisting of alkyl acrylates, alkyl methacrylates and vinyl esters of C₁-C₂₄ carboxylic acids, and
ii) 0.05 to 5% by weight of a monoolefinically unsaturated C₃-C₈ carboxylic acid.

3. A dispersion according to claim 1 where the monomer mixture consists essentially of:
i) 95 to 99.95% by weight of at least one monomer selected from the group consisting of ethyl acrylate, and methyl methacrylate,
ii) 0.05 to 5% by weight, of a monoolefinically unsaturated C₃-C₈ carboxylic acid, where the polymer dispersion in powder form is used as a coating agent for pharmaceutical dosage forms.

4. An aqueous polymer dispersion comprising polymers of comprising a monomer mixture wherein the monomer mixture consists of
i) 95 to 99.95% by weight of one or more monomers selected from the group consisting of alkyl acrylates and alkyl methacrylates, where the ratio of alkyl acrylates to alkyl methacrylates is from 9:1 to 1:3 by weight, and
ii) 0.05 to 5% by weight of a monoolefinically unsaturated C₃-C₈ carboxylic acid, and 0.001% to 1.0% by weight of an anionic emulsifier, based on the total weight of the monomers.

5. A dispersion according to claim 2, where the ratio of alkyl acrylates to alkyl methacrylates is from 3:1 to 1:1 by weight.

6. A dispersion according to claim 3, where the ratio of alkyl acrylates to alkyl methacrylates is from 2.5:1 to 1.5:1 by weight.

7. A dispersion according to claim 1, where ethyl acrylate and methyl methacrylate are employed as monomers i).

8. A dispersion according to claim 1, where methacrylic acid is employed as monomer ii).

9. A dispersion according to claim 1, where the anionic emulsifier comprises one or more compounds selected from the group consisting of ammonium salts or alkali metal salts of C₈-C₃₀ fatty acids, alkyl sulfates, alkylsulfonates or alkylarylsulfonates, where the alkyl radicals have 8 to 30 carbon atoms, sulfosuccinic monoesters and diesters and sulfated fatty alcohol ethoxylates.

10. A process for preparing aqueous dispersions according to claim 1 by emulsion polymerization in water.

11. A polymer in powder form, obtained from an aqueous dispersion according to claim 1.

12. The use of aqueous dispersions or polymers in powder form according to claim 11 as coating agent for pharmaceutical dosage forms.

13. The use according to claim 12, where the polymer dispersion further comprise one or more pharmaceutically acceptable excipients selected from the group consisting of antifoams, fillers, colors, pigments, antioxidants, preservatives, pore formers, gloss improvers and plasticizers.

14. A pharmaceutical dosage form with delayed release of active ingredient, comprising at least one active pharmaceutical ingredient, where the active ingredient is coated with a film coating obtained from an aqueous dispersion or a polymer according to claim 11.

15. A dispersion according to claim 4, where the ratio of alkyl acrylates to alkyl methacrylates is from 3:1 to 1:1 by weight.

16. A dispersion according to claim 4, where the ratio of alkyl acrylates to alkyl methacrylates is from 2.5:1 to 1.5:1 by weight.

17. An polymer dispersion comprising:
a monomer mixture, wherein the monomer mixture consists essentially of
i) 60 to 99.95% by weight of one or more monomers selected from the group consisting of alkyl acrylates, alkyl methacrylates and vinyl esters of C₁-C₂₄ carboxylic acids,
ii) 0.05 to 5% by weight of a monoolefinically unsaturated C₃-C₈ carboxylic acid, and
iii) 0 to 35% by weight of free-radical polymerizable monomers; and
0.001% to 1.0% by weight of an anionic emulsifier, based on the total weight of the monomers, where the polymer dispersion in powder form is provided in a pharmaceutical dosage form.

18. The polymer dispersion of claim 17, where the monomer mixture consists essentially of
i) 95 to 99.95% by weight of at least one monomer selected from the group consisting of alkyl acrylates, alkyl methacrylates and vinyl esters of C₁-C₂₄ carboxylic acids, and
ii) 0.05 to 5% by weight of a monoolefinically unsaturated C₃-C₈ carboxylic acid.

19. The polymer dispersion of claim 17, where the monomer mixture consists essentially of
i) 95 to 99.95% by weight of one or more monomers selected from the group consisting of alkyl acrylates and alkyl methacrylates, where the ratio of alkyl acrylates to alkyl methacrylates is from 9:1 to 1:3 by weight, and
ii) 0.05 to 5% by weight of a monoolefinically unsaturated C₃-C₈ carboxylic acid.

20. The polymer dispersion of claim 19, where the ratio of alkyl acrylates to alkyl methacrylates is from 3:1 to 1:1 by weight.

21. The polymer dispersion of claim 19, where the alkyl acrylates and alkyl methacrylates are ethyl acrylate and methyl methacrylate, respectively.

22. The polymer dispersion of claim 21, where the monoolefinically unsaturated C₃-C₈ carboxylic acid is methacrylic acid.

23. The use of the polymer dispersion of claim 17 in a coating for the pharmaceutical dosage form.
